(19) United States
(12) Patent Application Publication
(10) Pub. No.: US 2005/0032102 A1
(43) Pub. Date: Feb. 10, 2005

(54) Mapping genomic rearrangements

(75) Inventor: Ronald T. Shigeta, Berkeley, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/896,158

(22) Filed: Jul. 20, 2004

Related U.S. Application Data
(60) Provisional application No. 60/489,474, filed on Jul. 22, 2003.

(51) Int. Cl. C12Q 1/68; C12P 19/34

(52) U.S. Cl. 435/6; 435/91.2

(57) ABSTRACT

Methods and kits for monitoring genomic changes in an organism are provided. The complexity of a genomic sample is reduced in a reproducible manner and hybridized to an array of probes that are complementary to the genome of the organism. The hybridization pattern is compared to another hybridization pattern from a closely related organism to identify differences. Differences are indicative of possible rearrangements between the two genomes. The array may contain probes which are specifically designed to interrogate the presence or absence of specific regions of a genome or to interrogate regions of the genome at a relatively constant interval, such as about every 500 base pairs.

### BACKGROUND OF THE INVENTION

The genomes of a variety of organisms have been sequenced in recent years, including the genomes ranging from the human genome to the genomes of numerous bacteria and viruses. The recent completion of the sequence of the entire genome of a variety of different bacteria (and archaea) will allow researchers to study these genomes in a much more complete and rapid manner. Genomic rearrangement by recombination is a mechanism to generate diversity in genomes. The genomes of bacteria and other microorganisms undergo rearrangements resulting from homologous and non-homologous recombination. Rearrangements may result in an adaptive advantage to an organism, for example, antibiotic resistance and are of interest. Rearrangements may also account for speciation.

### SUMMARY OF THE INVENTION

A method is disclosed for detecting at least one genomic rearrangement between a first and second genomic sample. In one embodiment a first genomic sample is fragmented with a restriction enzyme thereby generating a population of fragments of heterogeneous size; the population of fragments is amplified so that a subset of target fragments is enriched relative to non-target fragments. The amplification product is hybridized to an array of probes wherein the array of probes comprises probes to one or more target fragments and hybridization pattern is compared to a second hybridization pattern from a second genomic sample to detect at least one difference between the two hybridization patterns. A difference may indicate that a genomic rearrangement has occurred.

In one embodiment the methods are used to map the location of a genomic rearrangement. The methods may be used to generate a map of differences between two species, two clones, two strains, or two isolates of a microorganism.

In one embodiment the hybridization pattern is detected using an array of probes. The array of probes is designed to detect genomic regions from an organism. The array may be designed using a reference genome. The probes of the array may hybridize to selected target fragments and may hybridize near the ends of target fragments. For example, the array may be designed to detect fragments from a specific species of E. coli and may be designed to hybridize to fragments of that genome that are 400 to 800 base pairs when that genome is digested with XbaI. The probes of the array may be designed to hybridize near an end of the fragments. For example, within 50, 100 or 200 base pairs of the end of the fragments.

Comparisons may be made, for example, between different clones, different species, or different isolates. Unknown isolates may be compared to one or more known isolates to, for example, identify an unknown isolate.

In one embodiment a method of detecting genomic rearrangement is disclosed. A subset of a genomic sample is separated from the genomic sample using a reproducible method such that when the method is used approximately the same subset is obtained each time. The subset is hybridized to an array of probes to generate an experimental hybridization pattern; and changes in the experimental hybridization pattern from an expected hybridization pattern or from a second experimental hybridization pattern are detected. The step of reproducibly separating a subset of a genomic sample from the genomic sample may be by amplifying the genomic sample by PCR wherein fragments of a selected size range are preferentially amplified so that fragments in the selected size range are enriched in the amplified sample relative to fragments that are longer or shorter than the selected size range. The size range may be 0, 200, 400, 1000, or 2000 to 800, 1000, 2000 or 5000 base pairs.

In some embodiments two clones are compared. The clones may be two different isolates of the same species of bacteria or two closely related microorganisms. In one embodiment one clone is derived from the first clone. One clone may be, for example, a descendant of the other clone, for example, an ancestral clone and a clone that has evolved from the ancestral clone through multiple generations of growth.

The method may be applied to any organism but is particularly suited to microorganisms, including bacteria, protozoa, fungi, and viruses.

In one embodiment a method of observing or monitoring evolution is disclosed. A genomic sample is isolated from a first clone of an organism as an ancestral sample and the ancestral clone or strain is subjected to multiple generations of growth to generate an evolved sample. The growth conditions may be modified to select for mutation or to add stress to the system. A second genomic sample is isolated from the evolved sample after the desired number of generations and a subset of fragments from the first and second samples are isolated using similar conditions. The samples are each hybridized to an array of probes to generate a hybridization pattern for each sample. The hybridization patterns are compared to identify at least one difference. A difference is an indication of a genomic change between the ancestral and the evolved sample.

### BRIEF DESCRIPTION OF THE FIGURES

FIG. 1a shows an example of a region of the genome with 5 XbaI restriction sites labeled Xba¹-Xba⁵. The sites that are recognized by probes on an array are indicated by a closed circle and the letters A-F. FIG. 1b shows the region in the absence of rearrangement and FIG. 1c shows the genomic segment after the rearrangement i.e. an inversion of a segment delineate by the closed and open triangle. Open circle surrounding the closed circle indicate the probes that will be detected on an array.

FIG. 2 shows the results of analysis of genomic DNA without rearrangement (FIG. 2a) and with rearrangement (FIG. 2b). The differences between the hybridization patterns identify regions that are likely to contain rearrangements.

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598 (United States Publication Number 2003003669).

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 (United States Publication Number 20040012676) and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194 (United States Publication Number 20040012676), 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621 (United States Publication Number 20030097222), 10/063,559 (United States Publication Number 20020183936), 10/065,856 (United States Publication Number 20030100995), 10/065,868 (United States Publication Number 20030120432), 10/328,818 (United States Publication Number 20040002818), 10/328,872, 10/423,403 (United States Publication Number 20040049354), and 60/482,389.

b) Definitions

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “clone” refers to a group of organisms or other living matter with the same genetic material. Many examples of clones exist in nature. Single-celled organisms, including bacteria, protozoa, and yeast, produce genetically identical offspring through asexual reproduction. These offspring develop from only one parent and are considered clones. Often microorganisms can be diluted so that a culture or colony may be started from a single individual. Each of the offspring in the culture or colony is a descendant of the first individual. These will be genetically identical except for new mutations that arose in subsequent generations and form a clone.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a lcolumn by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization conditions” as used herein will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage disequilibrium” or sometimes refer by allelic association as used herein refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

A term “microorganism” refers to a small organism, typically only seen with the aid of a microscope. Microbes include, but are not limited to, bacteria (both eubacteria and archeabacteria), fungi, protozoa and viruses.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population, but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

c. Oligonucleotides Array for Mapping Genomic Rearrangements via Homologous Recombination

Bacteria are known to adapt and to even form different strains by the process of genome rearrangement by, for example, homologous recombination, site specific recombination, transposition, translocation and non-homologous recombination. For example, a segment of the genome may be excised and reinserted in the same place but in an opposite orientation, for example, single-gene inversion. A comparison of two closely related organisms reveals that gene switching during divergence of the species occurred to positions that were similar distances from the origin of replication, but in the reverse direction. In one embodiment a method for detecting rearrangements or deletions using a complexity reduction method and an array of probes is disclosed.

Additional information about recombination may be found in Lewin, Genes VI 1997 Oxford University Press, New York, N.Y., which is incorporated herein by reference in its entirety and particularly in Chapter 17. See also Molecular Genetics of Bacteria, L. Snyder and W. Champness, 2^{nd} Edition, American Society for Microbiology, December 2002 and Bacterial and Bacteriophage Genetics, E. Birge, Springer Verlag, 2000, each of which is incorporated herein by reference in its entirety. See also Vulic et al., Proc. Natl. Acad. Sci. USA. 1999 June 22; 96 (13): 7348-7351.

Methods for detecting regions of recombination and rearrangement are disclosed. In some embodiments the methods include a step that reduces the complexity of a genomic sample in a reproducible way that depends on the length of restriction fragments and a method to detect the nucleic acids present in the reduced complexity sample using a high density array of oligonucleotide probes. The complexity reduction method is preferably reproducible and the detection method is preferably reproducible so that nucleic acids that are detected in one sample but not in another are indicative of rearrangement in one genome relative to the other. In many embodiments the complexity reduction method is a Whole Genome Sampling Assay (WGSA, see Kennedy et al., Nature Biotechnology, 2003; 21(10):1233-7 and U.S. Pat. No. 6,361,947 and US Patent Publication Nos. 20030096235, 20030082543 and 2003003669) and the method of detection is an array of probes. In an exemplary embodiment, the genomic DNA sample is extracted from an organism and digested with at least one restriction enzyme, adaptor sequences are attached to the restriction fragments, by for example, ligation, as described in U.S. Pat. No. 6,107,023. A generic primer that recognizes the adaptor sequence is used to amplify ligated DNA fragments. In a preferred embodiment, the PCR reaction is optimized to reproducibly amplify fragments of a particular size range, for example in the 200-2000 bp size range.

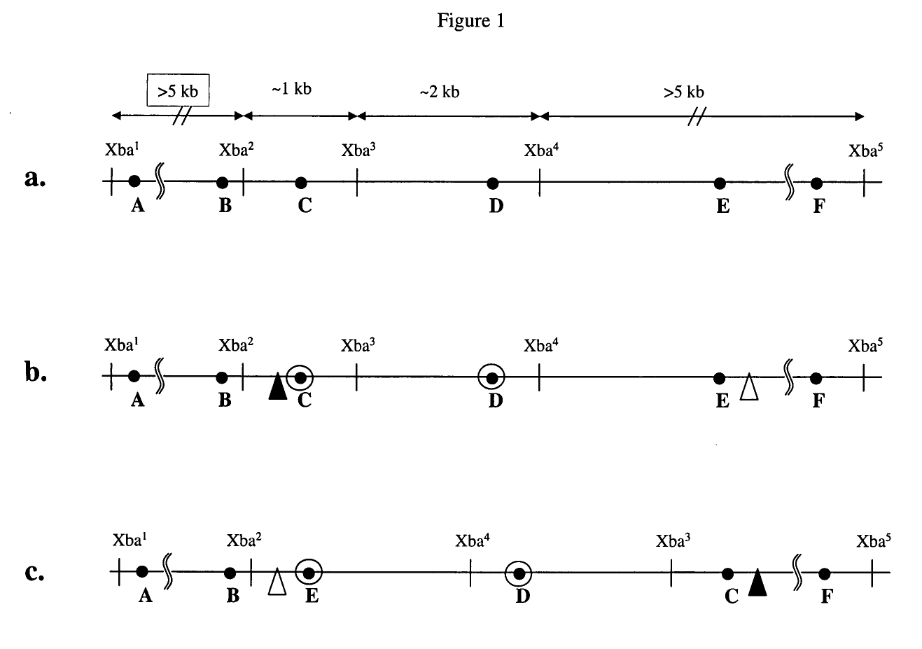

A schematic of one embodiment of the method is shown in FIG. 1. FIG. 1a shows a genomic segment with 5 XbaI restriction sites labeled Xba¹-Xba⁵. An XbaI digest will cut the fragment into 4 fragments, the first is greater than 5000 bp, the second is approximately 1000 bp, the third approximately 2000 bp and the fourth greater than 5000 bp. The fragments that are greater than 5000 in the example would not be detected because amplification of these large fragments would be inefficient. The fragments that are 1000 and 2000 bp would be detected because they are within the size range that will be amplified efficiently. The sites that are recognized by probes on an array are indicated by a closed circle and the letters A-F. In FIG. 1b the fragments that will be detected on the array in the absence of rearrangement are indicated by an open circle surrounding the closed circle of the probe that will detect the circle. C and D will be detected but A, B, E and F will not. The closed and open triangles indicate the boundaries of the rearrangement that is shown in FIG. 1c. FIG. 1c shows the genomic segment after the rearrangement indicated in 1B, the region delineated by the triangles has been flipped so that the regions detected by probe sets C, D and E are now in the order, E, D, C. As a result of the rearrangement, probes E and D are on fragments that are detectable size but C is on a large fragment that won't be amplified efficiently. The rearrangement between 1b and 1c may be detected by the disappearance of signal at the C probe set and appearance of signal at the E probe set. The A, B, D and F probe sets are not affected.

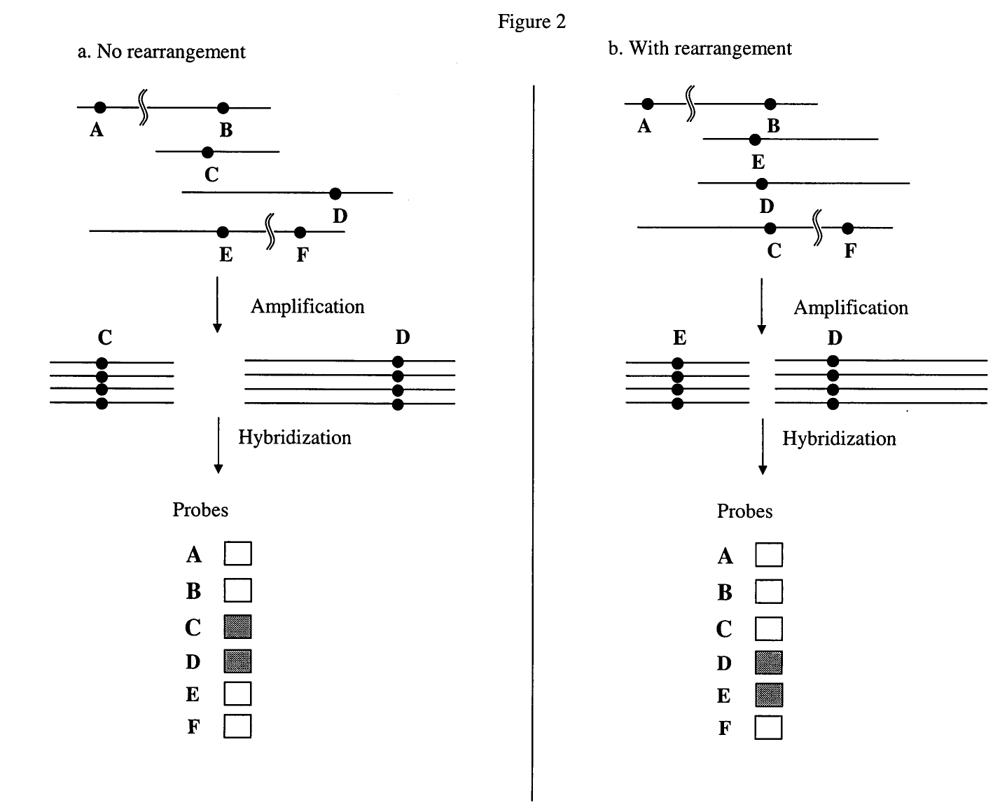

FIG. 2 shows a schematic of the overall process of one embodiment of the method. The fragments that would result from cleavage of the regions shown in FIGS. 1a and 1c are shown, 1a represents no rearrangement and 1c represents rearrangement. The fragments are amplified under conditions that efficiently amplify fragments that are less than about 2 kb but do not efficiently amplify larger fragments. The fragments containing regions complementary to probe sets C and D are efficiently amplified without rearrangement, but the fragments containing regions complementary to probes A, B, E and F are not efficiently amplified. Following hybridization signal is detected for probes C and D but not for probes A, B, E and F. For the sample that has undergone rearrangement (FIG. 2b) fragments containing E and D are efficiently amplified and following hybridization signal is detected for probes D and E but not for probes A, B, C and F.

Absence of hybridization may result, for example, from deletion of the region, insertion of extra sequence into a restriction fragment so that the restriction fragment is too long to be efficiently amplified, mutation in a restriction site that results in loss of that site so that a region that was previously amplified and detected because it was two smaller restriction fragments is now a single restriction fragment that is too big to be amplified, and rearrangement so that a region that was part of a fragment that was amplified is now part of a fragment that is too big to be amplified. Likewise, the presence of hybridization in a strain or clone that is absent in the ancestral clone, may result, for example, from deletion of a region so that a fragment that was previously too large to amplify is now small enough to be amplified (less than about 2 kb for example), a mutation that generates a new restriction site within a fragment that was previously too large to amplify, and rearrangement so that a region that was previously part of a fragment that was too big to be amplified is now part of a fragment that is small enough to be amplified. Secondary analysis methods known to those of skill in the art may be used to distinguish between the possible causes of a change.

In one embodiment a target subset of genomic DNA is separated from non-target genomic DNA or enriched relative to non-target DNA. The method of separation is preferably reproducible so that changes in the target subset may be attributed to changes in the genome. In one embodiment the method of separation is fragmentation with a selected restriction enzyme or enzymes and separation according to size by, for example, separation by gel electrophoresis, capillary electrophoresis or separation on a column. In another embodiment target fragments are enriched in the sample relative to non-target fragments. Amplification methods that preferentially amplify a reproducible subset of fragments over other fragments may be used. For example, PCR amplification typically favors amplification of fragments that are less than about 2500 basepairs and more preferably less than 2000 base pairs. Any amplification method that results in amplification of a reproducible subset of fragments may be used. Other PCR based amplification methods, for example, Degenerate Oligonucleotide Primer-PCR (DOP-PCR), see Telenius et al., Genes Chromosomes Cancer, 1992, 4(3):257-63, amplified fragment length polymorphisms (AFLP), see Vos et al. Nucleic Acids Res. 1995, 23(21):4407-14 and U.S. Pat. No. 6,045,994, or Arbitrarily primed PCR (AP-PCR), see Welsh and McClelland Nucleic Acids Res. 1990, 18(24):7213-8 may be used. Methods such as AFLP have been used to characterize closely related organisms using DNA fingerprinting methods that are related to the disclosed methods, see for example, U.S. Pat. No. 6,045,994.

In one embodiment genomic DNA from an organism is digested with one or more restriction enzymes to generate genomic fragments. A subset of the fragments is amplified. In one embodiment PCR is used to amplify the fragments and fragments of a selected size range, such as, for example fragments that are between 200 and 1000 base pairs, are amplified preferentially. In silico digestion of the genome is used to predict which fragments will fall within the selected size range given the enzyme or enzymes used for digestion and an array is designed and synthesized with probes that hybridize to those fragments. The array is capable of detecting the presence or absence of fragments in the selected size range following digestion of genomic DNA from a selected organism with selected enzymes. A restriction fragment is the DNA between two restriction enzyme recognition sites. If one of the sites moves then the resulting restriction fragment will be of a different size. If the restriction fragment increases in size so that it is outside the range of the fragments that are efficiently amplified by the amplification method that fragment may no longer be detected by hybridization to the array.

In one embodiment an array may be used to detect genome rearrangements. Genome rearrangements may be detected as an alteration to the pattern of hybridization. The rearrangement may result in movement of a restriction site to a new location in the genome, resulting in an increase or decrease in the size of one or more restriction fragments. Restriction fragments that are within the selected size range without rearrangement may fall outside of the selected size range after rearrangement. These fragments would be detected by the array before rearrangement but would not be detected after rearrangement. Likewise, regions that are on large fragments that are not efficiently amplified before rearrangement may be on smaller fragments that are efficiently amplified after rearrangement. These fragments may be detectable after rearrangement whereas they were not detectable before rearrangement. For example if the method of amplification used results in efficient amplification of fragments that are between 200 and 2500 base pairs and a probe set detects a fragment that is 1000 base pairs then that fragment will be efficiently amplified and detected without rearrangement. If the region of the genome is rearranged so that this region of the genome is now on a fragment that is 5000 base pairs that fragment will not be efficiently amplified and the probes to that region will not efficiently detect a fragment. Rearrangement may result in loss of signal because rearrangement results in an increase in the size of the fragment carrying the detected region or a decrease in the size of the fragment carrying the detected region provided that the increase or decrease makes the fragment bigger or smaller than the size range that is amplified efficiently by the method of amplification.

In one embodiment the probes on the array hybridize to the predicted fragments near a restriction site. For example, if the array is designed to detect XbaI fragments that are 200 to 2000 base pairs from the E. coli genome and a rearrangement results in the movement of an XbaI site so that a fragment that was 700 base pairs is now 5000 base pairs, the 5000 base pair fragment will no longer be efficiently amplified and probes that hybridize near that XbaI site will no longer detect a fragment.

In one embodiment an array comprising probes that hybridize to regions that are separated by approximately 500 bases throughout the genome is used. The probes may be evenly spaced along one or more chromosomes, chromosomal regions or genomes. In some embodiments the probes may be spaced more densely, for example, approximately every 250, 100 or 25 bases, or even every base, and in some embodiments they may be spaced less densely, for example, approximately every 1000, 2000, 5000 or 10,000 bases. The array may be designed to hybridize to the genomes of one or more organisms. In some embodiments the array may have probes to the entire genome of one or more organisms. For a description of arrays that may be used in some embodiments see US Patent Pub. No. 20030157529 and Kapranov et al., Science 296: 916-919, 2002. Other types of arrays may also be used, for example, genotyping arrays and expression arrays. See, for example US Patent Pub. Nos. 20040067493, 20030186279 and 20030198983. Methods of using arrays to determine copy number are disclosed, for example, in U.S. patent application Ser. No. 10/712,616 which is incorporated herein by reference in its entirety.

In one embodiment differences between two related organisms are identified. Genomic samples are isolated from each of the organisms. The samples are fragmented and amplified under essentially identical conditions, for example. The amplicons are fragmented, labeled and each sample is hybridized to an array to generate a hybridization pattern for each organism. The hybridization patterns are compared to identify differences.

In one embodiment pathogenic strains of bacteria are detected. Two strains may be closely related but vary in pathogenicity, one strain may be pathogenic while the other is not or one strain may cause more severe disease than the other. In one embodiment a pathogenic strain is distinguished from a non-pathogenic strain by detection of a rearrangement, insertion or deletion that is associated with the pathogenic strain or with the non-pathogenic strain. In one embodiment rearrangements between the pathogenic and non-pathogenic strain are identified by the methods disclosed above. Each strain is fragmented with a selected restriction enzyme or enzymes, adaptors are ligated to the fragments and the fragments are amplified by PCR using a primer that is complementary to the adaptor. A subset of fragments is amplified which is characteristic of the strain. The subset of fragments is then detected by hybridization to an array of probes that are complementary to a plurality of regions of the genome of the pathogens to generate a hybridization pattern that is characteristic of the pathogen strain being analyzed.

In another embodiment adaptation of bacteria to systematic stress is monitored. See Elena and Lenski, Nat Rev Genet. 2003; 4(6):457-69 which is incorporated herein by reference. Microorganisms are well-suited for experimental studies of evolution owing to their rapid generations and large populations, as well as the wealth of molecular and genomic data that are available for many species. Genetic adaptation may be especially rapid when microbial populations are introduced into new environments. In one embodiment a population is allowed to evolve, by for example, growth for many generations in a defined environment. The evolved population is then compared to the ancestral population by the methods disclosed to identify regions of genomic rearrangement. Rates of rearrangement may also be analyzed. The effect of clonal interference may also be monitored.

In one embodiment a specific strain of bacteria is identified from a culture. Disease causing strains of bacteria may be identified, for example, for diagnosis or epidemiological applications. Genomic DNA may be isolated from a strain and a hybridization pattern may be generated by the methods disclosed. The hybridization pattern may be compared to hybridization patterns from known bacteria to identify the bacteria in culture.

In one embodiment the evolutionary course of bacteria in a culture may be monitored. In one embodiment the reproducibility of evolutionary outcomes may be analyzed by monitoring microbial populations that are founded by the same ancestor and placed in identical environments. The rate of rearrangements and the locations of rearrangements may be monitored to determine if the rates are constant and if the locations are similar or unrelated.

In another embodiment subpopulations of strains may be identified in a mixture of bacteria. Individual isolates of may be isolated from a mixture containing that may contain two or more subpopulations of a strain of microorganism. Each isolate may be analyzed to provide a hybridization pattern according to the methods disclosed. The hybridization patterns may then be compared to identify differences. Some isolates may have the same hybridization pattern and others may have different hybridization patterns. Each different hybridization pattern may represent a different subpopulation or clone.

In one embodiment the methods are used to distinguish an antibiotic resistant strain from a strain that is sensitive to an antibiotic. The antibiotic resistant strain may have a different hybridization pattern from the sensitive strain.

In some embodiments an array and reagents that are useful for the embodiments may be packaged into a kit. The kit may comprise and array of probes for a particular organism or set of organisms, reagents for WGSA, such as primer, ligase, a thermal stable polymerase, such as Pfx polymerase, a restriction enzyme, buffer for the restriction enzyme, PCR buffer, DNase, dNTPs, a labeling reagent, terminal transferase, and fragmentation buffer. The kit may comprise some or all of these reagents. Instructions for use may also be included.

### d. EXAMPLES

One preferred method involves reducing the complexity of an experimental genomic sample by a method that reproducibly amplifies a subset of fragments that are within a selected size range, for example WGSA, labeling the reduced complexity sample (RCS), hybridizing the reduced complexity sample to an array to generate a hybridization pattern that is characteristic of the experimental genomic sample and comparing the hybridization pattern to at least one other hybridization pattern to identify differences.

Preparation of Reduced Complexity Samples-Whole Genome Sampling Assay (WGSA)

To increase sample throughputs, procedures may be carried out in 96-well plates. For each sample 250 ng of genomic DNA may be digested with 10 U of Xba I (New England BioLabs) in a volume of 15 μL for 2 hours at 37° C. Following heat inactivation at 70° C. for 20 minutes, 0.25 μM of adaptor (5′phosphate—CTA GAG ATC AGG CGT CTG TCG TGC TCA TAA-3′, and 5′-ATT ATG AGC ACG ACA GAC GCC TGA TCT-3′ synthesized by Qiagen) may be ligated to the digested DNA with T4 DNA Ligase (New England BioLabs) in 25 μL for 2 hours at 16° C. The ligation may be stopped by heating to 70° C. for 20 minutes, and then diluted 4-fold with water. For each sample, four PCRs may be run using 10 μL of the diluted ligation reaction (25 ng of starting DNA) in 100 μL volumes containing 0.75 μM of primer (sense strand of adaptor), 0.25 mM dNTPs, 2.5 mM MgCl₂, 10 U AmpliTaq Gold® (Applied Biosystems), and PCR Buffer (Applied Biosystems). 35 cycles of PCRs may be done in either MJ DNA Engine Tetrad (MJ Research) or GeneAmp PCR System 9700 (Applied Biosystems) cyclers. The cycling program in the MJ Tetrads may be 95° C. denaturation for 20 seconds, 59° C. annealing for 15 seconds, and 72° C. extension for 15 seconds. The denaturation, annealing and extension times may each be increased to 30 seconds when using the GeneAmp cycler. As a check, 3 μL of PCR products may be visualized on 2% TBE agarose gels to confirm the size range of amplicons. PCR products from the four reactions may be combined and purified over MinElute 96 UF PCR Purification plates (Qiagen). PCR amplicons from the four 100 μL reactions may be recovered in 40 μL of EB buffer (Qiagen). PCR yields, based on absorbance readings at 260 nm, may be typically ˜30 μg. To allow efficient hybridization to the 25-mer oligonucleotides on the array, PCR amplicons may be fragmented with DNAse I (Amersham Biosciences). 0.24 U of DNAse I may be added to 20 μg of purified PCR amplicons in a 55 μL volume containing 50 mM potassium acetate, 20 mM Tris-acetate, 10 mM magnesium acetate, and 1 mM dithiothreitol for 30 minutes at 37° C., followed by heat inactivation at 95° C. for 15 minutes. Fragmentation products may be visualized on 4% TBE agarose gels. The 3′ ends of the fragmented amplicons may be biotinlyated by adding 143 μM of a proprietary DNA labeling reagent (Affymetrix) using Terminal Deoxynucleotidyl Transferase (Promega) in a 70 μL volume containing 100 mM cacodylic acid (pH 6.8), 0.1 mM dithiothreitol, and 1 mM CoCl₂ for 2 hours at 37° C., followed by heat inactivation at 95° C. for 15 minutes.

Characterization of a Genomic Sample after Multiple Generations

A starting bacterial strain may be exposed to multiple generations under specific growth conditions. Genomic DNA from the bacterial strain before (i.e. ancestral) and after (i.e. evolved) multiple generations is isolated and the complexity is reduced using WGSA. Briefly, the genomic DNA is fragmented with XbaI, an adaptor sequence is ligated to the fragments and the fragments are amplified by PCR using a primer complementary to the adaptor under conditions that preferentially amplify fragments that are 400 to 800 base pairs. Fragments that are less than about 200 bp or greater than about 2000 bp are amplified inefficiently. The amplified sample is then fragmented and the fragments are end-labeled. The labeled fragments are hybridized to an array. The array has probes that detect fragments of the ancestral genome or reference genome that are 400 to 800 base pairs after XbaI digestion. The hybridization pattern of the ancestral and the evolved clones are compared to identify probes that produce signal in the ancestral but not in the evolved or probes that produce signal in the evolved clone but not in the ancestral. Those probes that produce a differential hybridization pattern between the two clones are possible locations of genomic rearrangements that arose during the multiple generations of growth under the selected conditions.

These regions of the genome may be mapped to identify the location of the rearrangement in the ancestral or reference genome and the results may provide information about the function of the genes present in those regions.

### CONCLUSION

A rapid method to identify locations of genomic rearrangement, deletion or insertion in a genome is described. The methods may be particularly useful for monitoring microorganisms. A subset of the genome is amplified in a manner that is reproducible so that fragments that are missing from one sample may be detected as an indication of genomic rearrangement, insertion or deletion.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

1. A method of detecting at least one genomic difference between a first and second genomic sample, wherein said first and second genomic samples are from a microorganism, comprising:
fragmenting a first genomic sample with at least one restriction enzyme thereby generating a first population of target fragments of heterogenous size;
amplifying the first population of fragments to generate a first amplification product wherein fragments that are longer than a lower size limit and shorter than an upper size limit are preferentially amplified relative to fragments that are shorter than the lower size limit or longer than the upper size limit;
fragmenting said first amplification product and labeling the fragments with a detectable label to form a first labeled amplification product;
fragmenting a second genomic sample with said at least one restriction enzyme thereby generating a second population of target fragments of heterogenous size;
amplifying the second population of fragments to generate a second amplification product wherein more than 50% of the mass of the amplified product is composed of fragments that are longer than 200 base pairs and shorter than 2,500 base pairs;
fragmenting said second amplification product and labeling the fragments with a detectable label to form a second labeled amplification product;
providing an array of probes comprising at least 10,000 target probes wherein each target probe is complementary to a different region of a reference genome;
generating a first and a second hybridization pattern by hybridizing the first labeled amplification product to a first copy of the array and the second labeled amplification product to a second copy of the array and detecting hybridization; and,
comparing the first hybridization pattern to the second hybridization pattern to detect at least one target probe that shows differential hybridization wherein differential hybridization is the presence of hybridization above background in one hybridization pattern and absence of hybridization above background in the other hybridization pattern.

2. The method of claim 1 wherein the target probes are complementary to non-repetitive regions of the genome.

3. The method of claim 1 wherein the reference genome is the genome of a microorganism.

4. The method of claim 3 wherein the microorganism is a bacterium.

5. The method of claim 1 wherein the target probes are evenly spaced along an entire genome of an organism.

6. The method of claim 5 wherein the target probes are spaced at about 5000 base pair intervals along a genomic region.

7. The method of claim 5 wherein the target probes are spaced at about 2000 base pair intervals along a genomic region.

8. The method of claim 5 wherein the target probes are spaced at about 1000 base pair intervals along a genomic region.

9. The method of claim 5 wherein the target probes are spaced at about 100 base pair intervals along a genomic region.

10. The method of claim 5 wherein the target probes are spaced at about 25 base pair intervals along a genomic region.

11. The method of claim 5 wherein the target probes are spaced every base pair along a genomic region.

12. The method of claim 1 wherein the target fragments are amplified by the polymerase chain reaction.

13. The method of claim 1 wherein each target probe is complementary to a different target fragment.

14. The method of claim 1 wherein the target fragments that will be preferentially amplified are predicted by simulated digestion of a reference genome using a computer system.

15. The method of claim 13 wherein at least 90% of the target probes are complementary to a region that is within 50 base pairs of an end of a target fragment.

16. The method of claim 13 wherein at least 90% of the target probes are complementary to a region that is within 100 base pairs of an end of a target fragment.

17. The method of claim 1 wherein the first and second genomic samples are from a first and a second clone of a first species.

18. The method of claim 17 wherein the second genomic sample is from an ancestral clone of the first species.

19. The method of claim 1 wherein the first and second genomic samples are from a first and second isolate of a microorganism.

20. The method of claim 19 wherein the first and second isolates have different pathogenicity.

21. The method of claim 19 wherein the first isolate is resistant to a first antibiotic and the second isolate is sensitive to the first antibiotic.

21. The method of claim 1 wherein the first and second genomic samples are from different species.

22. The method of claim 1 wherein the lower size limit is about 200 base pairs and the upper size limit is about 2,000 base pairs.

23. The method of claim 1 wherein the lower size limit is about 400 base pairs and the upper size limit is about 1,000 base pairs.

24. The method of claim 1 further comprising mapping a genomic rearrangement between a first and second genome to a region of a reference genome by determining the location in the reference genome of the at least one target probe that shows differential hybridization.

25. A method of detecting at least one genomic rearrangement between a first genomic sample from a microorganism and a reference sample comprising:
fragmenting the first genomic sample with at least one restriction enzyme thereby generating a first population of target fragments of heterogenous size;
amplifying the first population of fragments to generate a first amplification product wherein more than 50% of the mass of the amplified product is composed of fragments that are longer than 200 base pairs and shorter than 2,500 base pairs;
fragmenting said first amplification product and labeling the fragments with a detectable label to form a first labeled amplification product;
providing an array of probes comprising at least 10,000 target probes wherein each target probe is complementary to a different region of a reference genome;
generating an experimental hybridization pattern by hybridizing the first labeled amplification product to the array and detecting hybridization;
obtaining a reference hybridization pattern for the reference sample, and,
comparing the experimental hybridization pattern to the reference hybridization pattern to detect at least one target probe that shows differential hybridization wherein differential hybridization is the presence of hybridization above background in one hybridization pattern and absence of hybridization above background in the other hybridization pattern.

26. The method of claim 25 wherein the reference hybridization pattern is generated by predicting a hybridization pattern for the reference sample to the array.

27. The method of claim 25 wherein the reference sample is from a microorganism.

28. The method of claim 27 wherein the microorganism is a bacterium.

29. The method of claim 27 wherein the first genomic sample and the reference samples are from different clones of the same species.

30. The method of claim 25 wherein the first genomic sample is a genomic sample from a microorganism isolated from a human patient.

31. A method of observing evolution comprising:
isolating a genomic sample from a first clone of a microorganism as an ancestral sample;
subjecting the first clone to multiple generations of growth to generate an evolved sample;
isolating a second genomic sample from the evolved sample;
fragmenting the ancestral sample with at least one restriction enzyme thereby generating a first population of target fragments of heterogenous size;
amplifying the first population of fragments to generate a first amplification product wherein more than 50% of the mass of the amplified product is composed of fragments that are longer than 200 base pairs and shorter than 2,500 base pairs;
fragmenting the evolved sample with said at least one restriction enzyme thereby generating a second population of target fragments of heterogenous size;
amplifying the second population of fragments to generate a second amplification product wherein fragments that are longer than a lower size limit and shorter than an upper size limit are preferentially amplified relative to fragments that are shorter than the lower size limit or longer than the upper size limit;
providing an array of probes comprising at least 10,000 target probes wherein each target probe is complementary to a different region of a reference genome of the organism;
generating a first and a second hybridization pattern by hybridizing the first amplification product to a first copy of the array and the second amplification product to a second copy of the array and detecting hybridization; and,
comparing the first hybridization pattern to the second hybridization pattern to detect at least one target probe that shows differential hybridization wherein differential hybridization is the presence of hybridization above background in one hybridization pattern and absence of hybridization above background in the other hybridization pattern.
comparing the first and the second hybridization patterns to identify at least one difference in the hybridization pattern wherein the difference is indicative of a genomic change between the ancestral and the evolved sample.

32. The method of claim 31 wherein the organism is a microorganism.

33. The method of claim 32 wherein the organism is a bacterium.
